(19) United States
(12) Patent Application Publication
(10) Pub. No.: US 2015/0315539 A1
(43) Pub. Date: Nov. 5, 2015

(54) METHOD FOR STIMULATING CELL GROWTH

(71) Applicant: ALPHA THETA CENTER, INC., San Diego, CA (US)

(72) Inventors: Michael Villanueva, San Diego, CA (US); Gail K. Naughton, San Diego, CA (US)

(21) Appl. No.: 14/704,756

(22) Filed: May 5, 2015

Related U.S. Application Data
(60) Provisional application No. 61/988,792, filed on May 5, 2014.

(51) Int. Cl. C12N 5/00; A61N 2/00; A61N 5/06; A61N 2/02

(52) U.S. Cl. CPC C12N 5/0018; A61N 2/02; A61N 2/002; A61N 5/0613; A61N 2005/067

(57) ABSTRACT

The invention provides a method for stimulating cell growth, differentiation and/or protein production in a cell. The method includes contacting a cell growth medium or fluid with a pulsed electromagnetic field (pEMF) and a low level laser (LLL). Contacting may be sequential or simultaneous.

### CROSS-REFERENCE TO RELATED APPLICATIONS

This application claims benefit of priority under 35 U.S.C. §119(e) of U.S. Ser. No. 61/988,792, filed May 5, 2014, the entire contents of which is incorporated herein by reference in its entirety.

### BACKGROUND OF THE INVENTION

1. Field of Invention

The present invention relates to biotechnology, and more specifically to a method of stimulating cell growth, differentiation and/or protein production in a cell by application of radiation, e.g., electromagnetic radiation and pulsed electromagnetic fields, as well as compositions generated via the method.

2. Background Information

Pulsed electromagnetic field (pEMF) therapy has been used clinically for a number of indications, including speeding bone healing of non-union fractures, alleviating post-operative pain, and treating a variety of mental disorders such as depression, ADHD as well as sustained neurological symptoms associated with traumatic brain injury and Parkinson's disease. In the case of orthopedic indications the mechanism of action is thought to be accelerated cellular repair, in the case of psychiatric and neurological disorders the mechanism of action is thought to be energized constellations of neural assemblies more efficiently communicating. The output of pEMF devices varies greatly from less than 1-3 milligauss (used in testing cellular regeneration) to 10,000-15,000 Gauss typically used in rTMS and fMRI machines. However, using low output pEMF (typically 1-3 milligauss) for cellular research makes it possible to pulse and sustain EMF stimulation for longer periods of time without generating heat within the cells, thereby avoiding tissue damage. FDA has approved several pEMF stimulation devices.

With positive in vivo responses scientists have moved to look at the effect of pEMF on the growth of cells in vitro. All energy is electromagnetic in nature and every organ in the body—including structural components such as bone, tendons, and ligaments—produces its own signature bioelectromagnetic field, with all of the body's cells communicating via electromagnetic frequencies.

One research group has tested the effect of pEMF on the proliferation and differentiation potential of human bone marrow mesenchymal stem cells (MSCs). pEMF stimulus was administered to MSCs for 8 hours/day and consisted of 4.5 ms bursts repeating at 15 Hz with each burst containing 20 pulses. Results of this study showed that pEMF could enhance cell proliferation during the exponential phase as well as multilineage differentiation.

Considerable research has been performed to study how pEMF could affect the modulation of osteogenesis with MSCs. One research group exposed human MSCs in osteogenic media to daily pEMF stimulation with single 300 us quasi rectangular pulses with a repetition rate of 7.5 Hz for 28 days. Based on osteogenic markers and the upregulation of bone specific genes it was determined that low frequency pEMF increased the increased the production of bone by day seven. Similar results were seen by another group who demonstrated that pEMF enhanced the osteogenic effects of BMP-2 on MSCs cultured on calcium phosphate substrates.

The effect of pEMF on pluripotential embryonic stem cells (ES) was previously studied. Cells exposed to pEMF at 1.71 GHz showed a significant upregulation of mRNA for several proteins, including heat shot protein, p21 and c-myc. These studies support the use of pEMF to optimize cell growth and differentiation and protein production in vitro.

Recently microwaves and laser radiation have been applied to activate the growth processes of biological systems. One research group developed a gene promoter system that can be activated using short pulses of red light and can be deactivated using short pulses of infrared light, but the properties of the light and the pulses to activate the system were not specified. From previous publications it is possible to observe that the wavelength, the intensity and the exposition time are the most important parameters to produce activation of biological systems. The present work used an infrared-pulsed laser diode with wavelength λ=904 nm and peak power of 6 W to investigate the activation or deactivation of wheat seeds. The pulse repetition rate used for this laser was 1500 Hz with pulse duration of 200 ns. Wheat seeds were supplied by the National Institute of Forest, Agriculture and Livestock researches of Mexico (INIFAP). Wheat variety was Nahuatl F2000, developed by the institute above mentioned and the International Maize and Wheat Improvement Center (CIMMYT). To investigate the effects of laser radiation on wheat seeds, the stem length and the root length of wheat seedlings were measured. Wheat seeds were irradiated with one of the three intensities: 0.9 mW/cm2, 1.8 mW/cm2 and 3.6 mW/cm2 for five different exposition times: 15 s, 30 s, 60 s, 120 s and 240 s. For each laser treatment 400 wheat seeds divided into groups of 25 seeds according to the International Seed Testing Association (ISTA 2009) were used. The same quantity of non-radiated seeds was used as a control. The growth tests were carried out under laboratory conditions and according to guidelines issued by the International Seed Testing Association (ISTA 2009). The growth data were analyzed using the ANOVA test to detect differences between average values. Average values were compared using Tukey test (multiple comparisons) to detect differences between control and treated seedlings.

Clinical use of pulsed continuous wave (CV) laser has expanded in popularity over the past decade with treatments routinely involving skin resurfacing, ocular repair, and wound healing. Photonic stimuli infuses cells in the treatment area with energy, resulting in the decrease of inflammation, and an increase in cell regeneration and blood flow. One group studied the effects of pulsed versus CW lasers in a rodent wound healing model. An Erchonia pulse laser (635 nm) was used in the experiment. The percentage of relative wound healing was 4.32 in 100 Hz, 3.21 in 200 Hz, 3.83 in 300 Hz, 2.22 in 400 Hz, 1.73 in 500 Hz and 4.81 in CW demonstrating the benefit of using a CW laser to accelerate wound healing.

Low level laser (LLL) therapy (LLLT), including continuous wave (CW) laser has also been utilized for the enhancement of the proliferation of various cultured cell lines including stem cells. Many articles, report that it produces higher rates of ATP, RNA, and DNA synthesis in stem cells and other cell lines. (x,y,z) Mainly, helium neon and gallium-aluminum-arsenide (Ga—Al—As) lasers are used for LLLT on cultured cells. The results of LLLT also vary according to the applied energy density and wavelengths to which the target cells are subjected, with an energy density value of 0.5 to 4.0 J/cm(2) and a visible spectrum ranging from 600 to 700 nm of LLLT being the most effective in enhancing the proliferation rate of various cell lines.

### SUMMARY OF THE INVENTION

The present disclosure is based on the seminal discovery that application of both pEMF and LLL to a fluid medium including cells, such as a cell culture medium, stimulates cell growth, differentiation, protein production, or any combination thereof.

Accordingly, in one aspect, the invention provides a method for stimulating cell growth, differentiation, protein production, or any combination thereof, in a cell. The method includes contacting a cell growth medium or fluid containing the cell with pEMF and LLL, thereby stimulating cell growth, differentiation, protein production, or a combination thereof.

In another aspect, the invention provides a method of culturing a cell. The method includes: a) culturing the cell in a culture media; and b) contacting the culture media with pEMF and LLL, wherein the contacting increases cell growth, differentiation, protein production, or any combination thereof, by the cell.

In various embodiments, the pEMF and LLL may be applied to a fluid medium sequentially or simultaneously, and they may also be applied for any number of varied durations. In some embodiments, the pEMF frequency is selected from about 1 Hz to 4673 Hz and the LLL frequency is selected from about 73 Hz to 4672 Hz.

In another aspect, the invention provides a composition which includes one or more isolated components a fluid medium including a cell to which pEMF and LLL have been applied.

In another aspect, the invention provides a pre-energized liquid. In various embodiments the liquid is pre-energized by contacting the liquid with pEMF and LLL.

In another aspect, the pre-energized liquid is used for stimulating growth, differentiation, protein production, or any combination thereof, in a cell.

In another aspect, the invention provides a method for delivery of pEMF and LLL to a fluid medium. Delivery may be performed by a propeller or through a bioreactor jacket.

In another aspect, the invention provides a method of treating a subject in need thereof, comprising applying pEMF and a low level laser LLL to tissue of the subject, thereby treating the subject.

### DETAILED DESCRIPTION OF THE INVENTION

Although pEMF, LLLT, and CW have been studied independently both in vitro and in vivo, simultaneous or sequential stimulation of cells has not been studied. The current invention teaches delivery of both pEMF and LLL (including CW) to cells to increase cell proliferation and protein production. Energy is delivered into liquid which will come into direct contact with cells either in vitro or in vivo. In embodiments, energy is delivered via appliances embedded in a propeller and/or dispersed via prism which continuously stirs cells in a bioreactor.

Before the present compositions and methods are further described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

In one aspect, the invention provides a method for stimulating cell growth, differentiation, protein production, or any combination thereof, in a cell. The method includes contacting a cell growth medium or fluid containing the cell with pEMF and LLL, thereby stimulating cell growth, differentiation, protein production, or a combination thereof.

In another aspect, the invention provides a method of culturing a cell. The method includes: a) culturing the cell in a culture media; and b) contacting the culture media with pEMF and LLL, wherein the contacting increases cell growth, differentiation, protein production, or any combination thereof, by the cell.

In various embodiments of the invention, pEMF and LLL is applied. pEMF and LLL may be applied to cells in vivo or in vitro, directly to the cells or to a cellular medium or fluid containing the cell. pEMF and LLL may be applied to a cell in a cellular medium or fluid or to a cellular medium or fluid prior to introduction of the cell to generate a pre-energized fluid. In various embodiments, pEMF and LLL may be applied simultaneously or sequentially in any order. For example, pEMF may be applied prior to or after LLL. Additionally, pEMF and LLL may be applied any number of times. For example, each may be applied 1, 2, 3, 4, 5, 6, 7, 8, 9, 10 times or more in any sequence throughout the duration of cell culture, prior to cell culture, or throughout the duration of treatment.

The inventors contemplate use of a wide variety of pEMF frequencies. In general, pEMF frequencies range from about 1 Hz to 4673 Hz. In various embodiments, sets of pEMF frequencies may be utilized and include, but are not limited to: 2-3 Hz and 290-294 Hz, 4-5 Hz and 582-586 Hz, 9-10 Hz and 1167-1169 Hz, 18-19 Hz and 2335 Hz-2337 Hz, 36 Hz-37 Hz and 4670-4673 Hz, 72 Hz-74 Hz and 144-147 Hz.

The inventors contemplate use of a wide variety of LLL frequencies. In general, LLL frequencies range from about 73 Hz to 4672 Hz. In various embodiments, LLL and CW stimuli include the following core frequencies (all in Hz): 292 Hz, 584 Hz, 2336 Hz, 4672 Hz, 73 Hz and 146 Hz.

Additionally, the inventors contemplate use of LLL and CW stimuli in addition to those core frequencies recited above that are the 1^{st}, 2^{nd}, 3^{rd}, 4^{th}, and 5^{th}, harmonic frequency of each core frequency recited as defined by:

f₁=v/λ₁=v/2L;

f₂=v/λ₂=2v/2L=2f₁.

f₃=v/λ₃=3v/2L=3f₁;

f₄=v/λ₄=4v/2L=4f; and

f₅=v/λ₅=5v/2L=5f.

pEMF and LLL stimuli maybe applied for various durations. In embodiments, the stimuli may be applied for short durations, e.g., less than a minute, or longer durations, e.g., more than a minute. As such, the stimuli may be applied for about 1, 10, 20, 30, 40, 50 or 60 seconds, or for about 1, 5, 10, 20, 30, 40, 50, 60 minutes or longer, such as 1, 2, 3, 4, 5, 6, 7, 8, 9, 10, 11, 12, 13, 14, 15, 16, 17, 18, 19, 20, 21, 22, 23 or 24 hours or more. In some embodiments, stimuli is applied continuously throughout the duration of cell culture or therapy.

As discussed herein, cell growth, differentiation and protein production may be stimulated in a variety of different cell types. Such cells may include stromal cells, such as fibroblasts. In various aspects, the cells can be stromal cells comprising fibroblasts, with or without other cells. In some embodiments, the cells are stromal cells including, but not limited to: (1) bone; (2) loose connective tissue, including collagen and elastin; (3) the fibrous connective tissue that forms ligaments and tendons, (4) cartilage; (5) the ECM of blood; (6) adipose tissue, which comprises adipocytes; and (7) fibroblasts.

Stromal cells can be derived from various tissues or organs, such as skin, heart, blood vessels, bone marrow, skeletal muscle, liver, pancreas, brain, foreskin, which can be obtained by biopsy (where appropriate) or upon autopsy.

In other embodiments, cells includes stem cells or progenitor cells, either alone, or in combination with any other cell types. Stem cells and progenitor cells include, by way of example and not limitation, embryonic stem cells, hematopoietic stem cells, neuronal stem cells, epidermal stem cells, and mesenchymal stem cells.

Additional cell types of the invention include as non-limiting examples, smooth muscle cells, cardiac muscle cells, endothelial cells, skeletal muscle cells, endothelial cells, pericytes, macrophages, monocytes, and adipocytes. Stromal cells may be derived from appropriate tissues or organs, including, by way of example and not limitation, skin, heart, blood vessels, bone marrow, skeletal muscle, liver, pancreas, and brain.

In various embodiments, the cell is selected from stem cells, osteoblasts, chondrocytes, fibroblastic cells (e.g., interstitial fibroblasts, tendon fibroblasts, dermal fibroblasts, ligament fibroblasts, cardiac fibroblasts, periodontal fibroblasts such as gingival fibroblasts, and craniofacial fibroblasts), myocyte precursor cells, cardiac myocytes, skeletal myocytes, smooth muscle cells, striated muscle cells, satellite cells, chondrocytes (e.g., meniscal chondrocytes, articular chondrocytes, discus invertebralios chondrocytes), osteocytes, endothelial cells (e.g., aortic, capillary, and vein endothelial cells), epithelial cells (e.g., keratinocytes, adipocytes, hepatocytes), mesenchymal cells (e.g., dermal fibroblasts, mesothelial cells, osteoblasts), adipocytes, neurons, glial cells, Schwann cells, astrocytes, podocytes, islet cells, enterocytes, odontoblasts, and ameloblasts.

As discussed herein, stimuli may be applied to cells that are being cultured to promote cell growth, differentiation and/or protein production. Application of pEMF and LLL stimuli maybe use in any known methods of cell culture known in the art. For example, the stimuli may be applied to cultures of free floating cells or cells being cultured on a substrate, for example culture on a two-dimensional or three-dimensional surface in a suitable growth medium.

Cultivation materials providing three-dimensional architectures may be referred to as scaffolds which may be formed of microcarriers. The three-dimensional support or scaffold used to culture cells may be of any material and/or shape that: (a) allows cells to attach to it (or can be modified to allow cells to attach to it); and (b) allows cells to grow in more than one layer (i.e., form a three dimensional tissue). In other embodiments, a substantially two-dimensional sheet or membrane or beads may be used to culture cells that are sufficiently three dimensional in form.

In some aspects, the three dimensional framework is formed from polymers or threads that are braided, woven, knitted or otherwise arranged to form a framework, such as a mesh or fabric. The materials may also be formed by casting of the material or fabrication into a foam, matrix, or sponge-like scaffold. In other aspects, the three dimensional framework is in the form of matted fibers made by pressing polymers or other fibers together to generate a material with interstitial spaces. The three dimensional framework may take any form or geometry for the growth of cells in culture.

A number of different materials may be used to form the scaffold. These materials include non-polymeric and polymeric materials. Polymers, when used, may be any type of polymer, such as homopolymers, random polymers, copolymers, block polymers, coblock polymers (e.g., di, tri, etc.), linear or branched polymers, and crosslinked or non-crosslinked polymers. Non-limiting examples of materials for use as scaffolds or frameworks include, among others, glass fibers, polyethylenes, polypropylenes, polyamides (e.g., nylon), polyesters (e.g., dacron), polystyrenes, polyacrylates, polyvinyl compounds (e.g., polyvinylchloride; PVC), polycarbonates, polytetrafluorethylenes (PTFE; TEFLON), thermanox (TPX), nitrocellulose, polysaacharides (e.g., celluloses, chitosan, agarose), polypeptides (e.g., silk, gelatin, collagen), polyglycolic acid (PGA), and dextran.

In some aspects, the framework or beads may be made of materials that degrade over time under the conditions of use. Non-limiting examples of biodegradable materials include, among others, polylactide, polyglycolide, poly(trimethylene carbonate), poly(lactide-co-glycolide) (i.e., PLGA), polyethylene terephtalate (PET), polycaprolactone, catgut suture material, collagen (e.g., equine collagen foam), polylactic acid, or hyaluronic acid. For example, these materials may be woven into a three-dimensional framework such as a collagen sponge or collagen gel.

In various aspects, the scaffold or framework material may be pre-treated prior to inoculation with cells to enhance cell attachment. For example, prior to inoculation with cells, nylon screens in some embodiments are treated with 0.1 M acetic acid, and incubated in polylysine, fetal bovine serum, and/or collagen to coat the nylon. Polystyrene could be similarly treated using sulfuric acid. In other embodiments, the growth of cells in the presence of the three-dimensional support framework may be further enhanced by adding to the framework or coating it with proteins (e.g., collagens, elastin fibers, reticular fibers), glycoproteins, glycosaminoglycans (e.g., heparan sulfate, chondroitin-4-sulfate, chondroitin-6-sulfate, dermatan sulfate, keratan sulfate, etc.), fibronectins, and/or glycopolymer (poly[N-p-vinylbenzyl-D-lactoamide], PVLA) in order to improve cell attachment. Treatment of the scaffold or framework is useful where the material is a poor substrate for the attachment of cells.

In some embodiments, the scaffold is composed of microcarriers, which are beads or particles. The beads may be microscopic or macroscopic and may further be dimensioned so as to permit penetration into tissues or compacted to form a particular geometry. In some tissue penetrating embodiments, the framework for the cell cultures comprises particles that, in combination with the cells, form a three dimensional tissue. The cells attach to the particles and to each other to form a three dimensional tissue. The complex of the particles and cells is of sufficient size to be administered into tissues or organs, such as by injection or catheter. Beads or microcarriers are typically considered a two-dimensional system or scaffold.

As used herein, a “microcarriers” refers to a particle having size of nanometers to micrometers, where the particles may be any shape or geometry, being irregular, non-spherical, spherical, or ellipsoid.

The size of the microcarriers suitable for the purposes herein can be of any size suitable for the particular application. In some embodiments, the size of microcarriers suitable for the three dimensional tissues may be those administrable by injection. In some embodiments, the microcarriers have a particle size range of at least about 1 μm, at least about 10 μm, at least about 25 μm, at least about 50 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1000 μm.

In some aspects in which the microcarriers are made of biodegradable materials. In some aspects, microcarriers comprising two or more layers of different biodegradable polymers may be used. In some embodiments, at least an outer first layer has biodegradable properties for forming the three dimensional tissues in culture, while at least a biodegradable inner second layer, with properties different from the first layer, is made to erode when administered into a tissue or organ.

In some aspects, the microcarriers are porous microcarriers. Porous microcarriers refer to microcarriers having interstices through which molecules may diffuse in or out from the microparticle. In other embodiments, the microcarriers are non-porous microcarriers. A nonporous microparticle refers to a microparticle in which molecules of a select size do not diffuse in or out of the microparticle.

Microcarriers for use in the compositions are biocompatible and have low or no toxicity to cells. Suitable microcarriers may be chosen depending on the tissue to be treated, type of damage to be treated, the length of treatment desired, longevity of the cell culture in vivo, and time required to form the three dimensional tissues. The microcarriers may comprise various polymers, natural or synthetic, charged (i.e., anionic or cationic) or uncharged, biodegradable, or nonbiodegradable. The polymers may be homopolymers, random copolymers, block copolymers, graft copolymers, and branched polymers.

In some aspects, the microcarriers comprise non-biodegradable microcarriers. Non-biodegradable microcapsules and microcarriers include, but not limited to, those made of polysulfones, poly(acrylonitrile-co-vinyl chloride), ethylene-vinyl acetate, hydroxyethylmethacrylate-methyl-methacrylate copolymers. These are useful to provide tissue bulking properties or in embodiments where the microcarriers are eliminated by the body.

In some aspects, the microcarriers comprise degradable scaffolds. These include microcarriers made from naturally occurring polymers, non-limiting example of which include, among others, fibrin, casein, serum albumin, collagen, gelatin, lecithin, chitosan, alginate or poly-amino acids such as poly-lysine. In other aspects, the degradable microcarriers are made of synthetic polymers, non-limiting examples of which include, among others, polylactide (PLA), polyglycolide (PGA), poly(lactide-co-glycolide) (PLGA), poly(caprolactone), polydioxanone trimethylene carbonate, polyhybroxyalkonates (e.g., poly(hydroxybutyrate), poly(ethyl glutamate), poly(DTH iminocarbony(bisphenol A iminocarbonate), poly(ortho ester), and polycyanoacrylates.

In some aspects, the microcarriers comprise hydrogels, which are typically hydrophilic polymer networks filled with water. Hydrogels have the advantage of selective trigger of polymer swelling. Depending on the composition of the polymer network, swelling of the microparticle may be triggered by a variety of stimuli, including pH, ionic strength, thermal, electrical, ultrasound, and enzyme activities. Non-limiting examples of polymers useful in hydrogel compositions include, among others, those formed from polymers of poly(lactide-co-glycolide); poly(N-isopropylacrylamide); poly(methacrylic acid-g-polyethylene glycol); polyacrylic acid and poly(oxypropylene-co-oxyethylene) glycol; and natural compounds such as chrondroitan sulfate, chitosan, gelatin, fibrinogen, or mixtures of synthetic and natural polymers, for example chitosan-poly (ethylene oxide). The polymers may be crosslinked reversibly or irreversibly to form gels adaptable for forming three dimensional tissues.

In exemplary aspects, the microcarriers or beads for use in the present invention are composed wholly or composed partly of dextran.

Culture of the cells may be performed using standard conditions which are known for a particular cell type. In some embodiments, culture may be performed under hypoxic conditions. As used herein, hypoxic conditions are characterized by a lower oxygen concentration as compared to the oxygen concentration of ambient air (approximately 15%-20% oxygen). In one aspect, hypoxic conditions are characterized by an oxygen concentration less than about 10%. In another aspect hypoxic conditions are characterized by an oxygen concentration of about 1% to 10%, 1% to 9%, 1% to 8%, 1% to 7%, 1% to 6%, 1% to 5%, 1% to 4%, 1% to 3%, or 1% to 2%. In a certain aspect, the system maintains about 1-3% oxygen within the culture vessel. Hypoxic conditions can be created and maintained by using a culture apparatus that allows one to control ambient gas concentrations, for example, an anaerobic chamber.

In various embodiments culture may be performed for any amount of time necessary for sufficient growth of the cells. For example, cells may be cultured for 1, 2, 3, 4, 5 or 6 days or more including 1, 2, 3, 4 or 5 weeks or more.

In various embodiments, cells may be incubated in an appropriate nutrient medium with incubation conditions that supports growth of cells, differentiation and protein synthesis. Many commercially available media such as Dulbecco's Modified Eagles Medium (DMEM), RPMI 1640, Fisher's, Iscove's, and McCoy's, may be suitable for supporting the growth of the cell cultures. The medium may be supplemented with additional salts, carbon sources, amino acids, serum and serum components, vitamins, minerals, reducing agents, buffering agents, lipids, nucleosides, antibiotics, attachment factors, and growth factors. Formulations for different types of culture media are described in various reference works available to the skilled artisan (e.g., Methods for Preparation of Media, Supplements and Substrates for Serum Free Animal Cell Cultures, Alan R. Liss, New York (1984); Tissue Culture: Laboratory Procedures, John Wiley & Sons, Chichester, England (1996); Culture of Animal Cells, A Manual of Basic Techniques, 4 th Ed., Wiley-Liss (2000)). The growth or culture media used in any of the culturing steps of the present invention, whether under aerobic or hypoxic conditions, may include serum, or be serum free.

Incubation conditions may be under appropriate conditions of pH, temperature, and gas (e.g., O₂, CO₂, etc) to maintain growth. In some embodiments, the culture may be “fed” periodically to remove the spent media, depopulate released cells, and add new nutrient source.

The presently described methods of the invention may further include detecting and/or harvesting of cells, cellular components, or secreted factors, such as proteins, hormones and steroids that are secreted during growth. As such, in a further aspect, the present invention provides compositions that include isolated components of cultured cellular media. This may include whole cells or components thereof. The compositions of the present invention may include both soluble and non-soluble fractions or any portion thereof of the cultured cellular media. It is to be understood that the compositions of the present invention may include either or both fractions, as well as any combination thereof. Additionally, individual components may be isolated from the fractions to be used individually or in combination with other isolates or known compositions.

Accordingly, in various aspects, compositions produced using the methods of the present invention may be used directly or processed in various ways, the methods of which may be applicable to both the non-soluble and soluble fractions. The soluble fraction, including the cell-free supernatant and media, may be subject to lyophilization for preserving and/or concentrating the factors. Various biocompatible preservatives, cryoprotectives, and stabilizer agents may be used to preserve activity where required. Examples of biocompatible agents include, among others, glycerol, dimethyl sulfoxide, and trehalose. The lyophilizate may also have one or more excipients such as buffers, bulking agents, and tonicity modifiers. The freeze-dried media may be reconstituted by addition of a suitable solution or pharmaceutical diluent, as further described below.

In other aspects, the soluble fraction is dialyzed. Dialysis is one of the most commonly used techniques to separate sample components based on selective diffusion across a porous membrane. The pore size determines molecular-weight cutoff (MWCO) of the membrane that is characterized by the molecular-weight at which 90% of the solute is retained by the membrane. In certain aspects membranes with any pore size is contemplated depending on the desired cutoff. Typical cutoffs are 5,000 Daltons, 10,000 Daltons, 30,000 Daltons, and 100,000 Daltons, however all sizes are contemplated.

In some aspects, the soluble fraction may be processed by precipitating the active components (e.g., growth factors) in the media. Precipitation may use various procedures, such as salting out with ammonium sulfate or use of hydrophilic polymers, for example polyethylene glycol.

In other aspects, the soluble fraction is subject to filtration using various selective filters. Processing the soluble fraction by filtering is useful in concentrating the factors present in the fraction and also removing small molecules and solutes used in the soluble fraction.

Filters with selectivity for specified molecular weights include <5000 Daltons, <10,000 Daltons, and <15,000 Daltons. Other filters may be used and the processed media assayed for therapeutic activity as described herein. Exemplary filters and concentrator system include those based on, among others, hollow fiber filters, filter disks, and filter probes (see, e.g., Amicon Stirred Ultrafiltration Cells).

In still other aspects, the soluble fraction is subject to chromatography to remove salts, impurities, or fractionate various components of the medium. Various chromatographic techniques may be employed, such as molecular sieving, ion exchange, reverse phase, and affinity chromatographic techniques. For processing conditioned medium without significant loss of bioactivity, mild chromatographic media may be used. Non-limiting examples include, among others, dextran, agarose, polyacrylamide based separation media (e.g., available under various tradenames, such as Sephadex™, Sepharose™, and Sephacryl™).

In another aspect, the invention provides a method of treating a subject in need thereof, comprising applying pEMF and a low level laser LLL to tissue of the subject, thereby treating the subject. The application of stimuli may be applied directly to the tissue of the subject or cells or tissue may be taken from the subject and stimuli applied in vitro. The cells, or a component thereof, may then be returned to the subject.

In another aspect, the invention provides a method of treating a subject in need thereof, comprising administering a composition of the present invention to the subject, thereby treating the subject.

The terms “administration” or “administering” are defined to include an act of providing a compound or pharmaceutical composition of the invention to a subject in need of treatment. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrastemal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The compositions or active components used herein, will generally be used in an amount effective to treat or prevent the particular disease being treated. The compositions may be administered therapeutically to achieve therapeutic benefit or prophylactically to achieve prophylactic benefit. By therapeutic benefit is meant eradication or amelioration of the underlying condition or disorder being treated. Therapeutic benefit also includes halting or slowing the progression of the disease, regardless of whether improvement is realized.

The amount of the composition administered will depend upon a variety of factors, including, for example, the type of composition, the particular indication being treated, the mode of administration, whether the desired benefit is prophylactic or therapeutic, the severity of the indication being treated and the age and weight of the patient, and effectiveness of the dosage form. Determination of an effective dosage is well within the capabilities of those skilled in the art.

Initial dosages may be estimated initially from in vitro assays. Initial dosages can also be estimated from in vivo data, such as animal models. Animals models useful for testing the efficacy of compositions for enhancing hair growth include, among others, rodents, primates, and other mammals. The skilled artisans can determine dosages suitable for human administration by extrapolation from the in vitro and animal data.

Dosage amounts will depend upon, among other factors, the activity of the conditioned media, the mode of administration, the condition being treated, and various factors discussed above. Dosage amount and interval may be adjusted individually to provide levels sufficient to the maintain the therapeutic or prophylactic effect.

Although the invention has been described, it will be understood that modifications and variations are encompassed within the spirit and scope of the invention. Accordingly, the invention is limited only by the following claims.

What is claimed is:

1. A method for stimulating cell growth, differentiation, protein production, or any combination thereof, in a cell comprising contacting a cell growth medium or fluid with a pulsed electromagnetic field (pEMF) and a low level laser (LLL), thereby stimulating cell growth, differentiation, protein production, or a combination thereof.

2. The method of claim 1, wherein contacting is performed sequentially.

3. The method of claim 1, wherein contacting is performed simultaneously.

4. The method of claim 1, wherein the cell growth medium or fluid is contacted a plurality of times.

5. The method of claim 1, wherein the pEMF frequency is selected from about 1 Hz to 4673 Hz.

6. The method of claim 1, wherein the LLL frequency is selected from about 73 Hz to 4672 Hz.

7. The method of claim 1, wherein the cell is selected from the group consisting of stem cells, osteoblasts, chondrocytes, fibroblastic cells, myocyte precursor cells, cardiac myocytes, skeletal myocytes, smooth muscle cells, striated muscle cells, satellite cells, chondrocytes, osteocytes, endothelial cells, epithelial cells, mesenchymal cells, adipocytes, neurons, glial cells, Schwann cells, astrocytes, podocytes, islet cells, enterocytes, odontoblasts, and ameloblasts.

8. The method of claim 1, further comprising culturing the cell.

9. The method of claim 8, wherein the cell is cultured under hypoxic conditions.

10. The method of claim 8, further comprising harvesting or isolating a component from the cell growth medium or fluid.

11. The method of claim 10, wherein the component is a cell or cellular component thereof.

12. The method of claim 11, wherein the component is a biological molecule selected from the group consisting of oligonucleotides and proteins.

13. The method of claim 8, further comprising detecting an increase in cell growth, differentiation or protein production.

14. A method of culturing a cell comprising:
a) culturing the cell in a culture media; and
b) contacting the culture media with a pulsed electromagnetic field (pEMF) and a low level laser (LLL), wherein the contacting increases cell growth, differentiation, protein production, or any combination thereof, by the cell.

15. The method of claim 14, wherein contacting is performed sequentially.

16. The method of claim 14, wherein contacting is performed simultaneously.

17. The method of claim 14, wherein the cell growth medium or fluid is contacted a plurality of times.

18. The method of claim 14, wherein the pEMF frequency is selected from about 1 Hz to 4673 Hz.

19. The method of claim 14, wherein the LLL frequency is selected from about 73 Hz to 4672 Hz.

20. The method of claim 14, wherein the cell is selected from the group consisting of stem cells, osteoblasts, chondrocytes, fibroblastic cells, myocyte precursor cells, cardiac myocytes, skeletal myocytes, smooth muscle cells, striated muscle cells, satellite cells, chondrocytes, osteocytes, endothelial cells, epithelial cells, mesenchymal cells, adipocytes, neurons, glial cells, Schwann cells, astrocytes, podocytes, islet cells, enterocytes, odontoblasts, and ameloblasts.

21. The method of claim 14, further comprising culturing the cell at least 1, 2, 3, 4, 5, 6, 7, 8, 9, 10 days or more.

22. The method of claim 21, wherein the cell is cultured under hypoxic conditions.

23. The method of claim 14, further comprising harvesting or isolating a component from the cell growth medium or fluid.

24. The method of claim 23, wherein the component is a cell or cellular component thereof.

25. The method of claim 24, wherein the component is a biological molecule selected from the group consisting of oligonucleotides and proteins.

26. The method of claim 14, further comprising detecting an increase in cell growth, differentiation or protein production.

27. A composition comprising the isolated component of claim 23.

28. A composition comprising a pre-energized liquid.

29. The composition of claim 28, wherein the liquid is water or a cell culture medium.

30. The composition of claim 28, further comprising one or more additives selected from the group consisting of: vitamins, supplements, growth media and polypeptides.

31. The composition of claim 30, wherein the additive is one or more polypeptides selected from the group consisting of: transcription factors, growth factors, enzymes, hormones and steroids.

32. The composition of claim 28, wherein the liquid is pre-energized by contacting the liquid with a pulsed electromagnetic field (pEMF) and a low level laser (LLL).

33. The composition of claim 32, wherein the pEMF frequency is selected from about 1 Hz to 4673 Hz.

34. The composition of claim 32, wherein the LLL frequency is selected from about 73 Hz to 4672 Hz.

35. The composition of claim 28, wherein the liquid further comprises a cell.

36. The composition of claim 35, wherein the cell is selected from the group consisting of stem cells, osteoblasts, chondrocytes, fibroblastic cells, myocyte precursor cells, cardiac myocytes, skeletal myocytes, smooth muscle cells, striated muscle cells, satellite cells, chondrocytes, osteocytes, endothelial cells, epithelial cells, mesenchymal cells, adipocytes, neurons, glial cells, Schwann cells, astrocytes, podocytes, islet cells, enterocytes, odontoblasts, and ameloblasts.

37. The use of a composition of claim 28 for stimulating growth, differentiation, protein production, or any combination thereof, in a cell.

38. A method for delivery of a pulsed electromagnetic field (pEMF) and a low level laser (LLL) to a fluid medium, wherein delivery is via a propeller.

39. The method of claim 38, wherein the propeller is in a bioreactor.

40. The method of claim 38, wherein the fluid medium is a cell culture medium.

41. A method for delivery of a pulsed electromagnetic field (pEMF) and a low level laser (LLL), wherein delivery is through a bioreactor jacket.

42. The method of any of claim 38 or 41, wherein delivery is sequential or simultaneous.

43. A method of treating a subject in need thereof, comprising applying a pulsed electromagnetic field (pEMF) and a low level laser (LLL) to tissue of the subject, thereby treating the subject.

44. The method of claim 43, wherein the subject is a mammal.

45. The method of claim 44, wherein the subject is a human.

46. The method of claim 43, wherein pEMF and LLL are applied sequentially or simultaneously.
